(19) United States
(12) Patent Application Publication
(10) Pub. No.: US 2023/0380034 A1
(43) Pub. Date: Nov. 23, 2023

(54) Modulated UVC LED Array Apparatus

(71) Applicant: James A. Allen, JR., Gilroy, CA (US)

(72) Inventor: James A. Allen, JR., Gilroy, CA (US)

(21) Appl. No.: 18/198,145

(22) Filed: May 16, 2023

Related U.S. Application Data
(60) Provisional application No. 63/343,241, filed on May 18, 2022.

(51) Int. Cl. H05B 45/325; H05B 45/46; H05B 45/36; H05B 47/105; A61L 2/10

(52) U.S. Cl. CPC H05B 45/325; H05B 45/46; H05B 45/36; H05B 47/105; A61L 2/10

(57) ABSTRACT

A modulated LED array apparatus includes a modulation signal generator that produces a pulse width modulation (PWM) signal that is applied as input to an LED power drive circuit that produces an LED power drive signal; the LED power drive signal is applied as input to an array of LEDs and, in response, the LED array outputs PWM radiation, wherein the LEDs in the array output radiation in a predetermined band of wavelength.

This patent application relates to U.S. Provisional Application No. 63/343,241 filed May 18, 2022 from which priority is claimed under 35 USC § 119(e), and which provisional application is incorporated herein in its entirety.

### TECHNICAL FIELD

One or more embodiments relate to modulated UVC LED array apparatus, and in particular, to FPWM modulated UVC LED array apparatus.

### BACKGROUND

Ultraviolet (“UV”) light encompasses three sub-bands which range in wavelength between 200 nm and 280 nm. These sub-bands are generally referred to as UVA, UVB, and UVC, where UVA has the longest wavelengths, followed by UVB and followed by UVC which has the shortest wavelengths. As is well known, UVC is germicidal, meaning it can be used as a disinfectant to kill microorganisms, such as bacteria and viruses. When the DNA of a microorganism absorbs UV light, the microorganism is stopped from being able to reproduce and duplicate, thereby preventing its growth. In the case of disinfection, an optimum wavelength is in a region from about 260 nm to about 270 nm, with germicidal efficacy falling exponentially with longer wavelengths.

Conventional UVC lamps have drawbacks such as, for example, they are not compact, they are fragile, and they use lots of energy. These conventional UV lamps require routine replacement, and they are susceptible to breakage during transportation, handling, and operation. In addition, conventional UV lamps typically use between 5-200 mg of mercury per lamp. Such conventional UV lamps hold their mercury either in a liquid form (more common in medium pressure lamps) or in an amalgam (more common in low pressure, high output lamps). Amalgam UV lamps contain solid “spots” which consist of an alloy of mercury and another element, such as indium or gallium. Liquid mercury lamps have the potential to be hazardous both during operation and when the lamp is off. While the lamp is operating, the mercury vaporizes; if the lamp is broken, mercury vapor is easily dissolved into the treated product. Accidents and improper procedures increase the risk of exposure to people and the local environment.

UVC LEDs offer several advantages over conventional UVC lamps. First, UVC LEDs are much more compact than their mercury-vapor counterparts. As such, they can be integrated readily into new designs. Second, UVC LEDs are not fragile. Third, UVC LEDs consume less energy than conventional UVC lamps. Fourth, UVC LEDs can be designed to have a peak emission that is closer to a germicidal absorption peak than conventional UVC lamps. In other words, UVC LEDs can be designed to choose specific wavelengths that are best suited for maximum absorption of light by a particular microorganism. FIG. 5 shows a germicidal effectiveness curve and the relative output from a low-pressure mercury lamp. As one can see from FIG. 5, output from the mercury lamp is displaced from the peak of germicidal effectiveness. Thus, it would be more effective to use output from UVC LEDs than from a mercury lamp for disinfection purposes. Fifth, UVC LEDs are monochromatic in the UVC region and do not generate ozone emission since ozone generation from UV light typically occurs below 240 nm. Sixth, UVC LED apparatus is mercury free and, thereby, provides a safer alternative to conventional mercury lamp apparatus. Seventh, UVC LEDs provide instant on/off operation. As such, there is no need for a warm-up time period that is a common constraint of mercury-vapor lamps. Eighth, UVC LEDs have unlimited cycling since on/off cycles do not impact the life of the LEDs. Ninth, although UVC LEDs do contain small amounts of elements such as the metals gallium and magnesium and the metalloids silicon and boron (although boron is not predominantly used), these metals and or metalloids are bound within a stable crystal structure and cannot leach into the environment. Tenth, UVC LEDs can be temperature independent meaning LEDs can be designed to emit photons from a different surface from their heat emissions. As such, UVC LEDs can be designed so that, if UVC LEDs are being used, for example, in water purification, they will not transfer heat into the water.

In light of the above, there is a need in the art for an improved UVC LED array apparatus.

### SUMMARY

One or more embodiments is a an FPWM modulated UVC array apparatus that comprises: a modulation signal generator that produces a pulse width modulation (PWM) signal that is applied as input to an LED power drive circuit that produces an LED power drive signal; the LED power drive signal is applied as input to an array of LEDs and, in response, the LED array outputs PWM radiation, wherein the LEDs in the array output radiation in a predetermined band of wavelength.

### BRIEF DESCRIPTION OF THE DRAWINGS

FIG. 1 shows a block diagram of an FPWM modulated UVC LED array apparatus that is fabricated in accordance with one or more embodiments.

FIG. 2 shows a cross section of a portion of an FPWM modulated UVC LED array apparatus that is fabricated in accordance with one or more embodiments wherein an LED array emits radiation into a cuvette through which a liquid passes, which cuvette is substantially transparent to the radiation.

FIG. 3 shows a schematic diagram of an FPWM signal generator that is fabricated in accordance with one or more embodiments.

FIG. 4 shows a schematic diagram of an LED array power driver that is fabricated in accordance with one or more embodiments.

FIG. 5 shows a superposed plot of mercury lamp emission and a bacteria's DNA absorption.

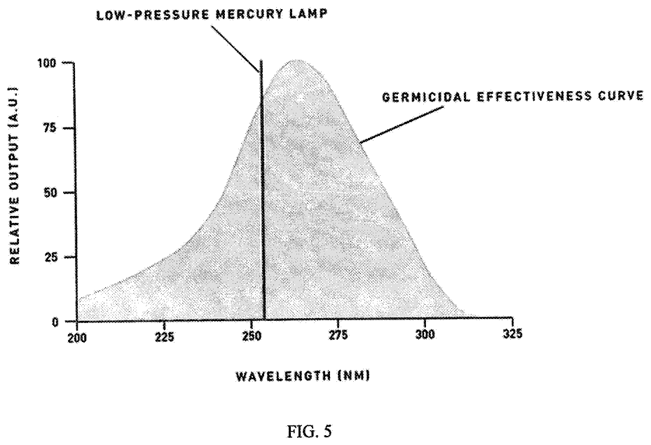

FIG. 6 shows a block diagram of a blood disinfection apparatus that includes an embodiment of the FPWM modulated LED array apparatus shown in FIG. 1.

### DETAILED DESCRIPTION

One or more embodiments comprise an apparatus that outputs radiation from an array of light emitting diodes (“LEDs”), wherein power input to the array of LEDs (referred to herein as a “LED drive signal”) is modulated. In accordance with one or more embodiments, the LED drive signal is modulated using fixed frequency, pulse width modulation (“FPWM”).

Many applications exist for using one or more embodiments of the FPWM modulated UVC LED array apparatus described herein, for example and without limitation, for pathogen disinfection by, for example, destroying a pathogen's DNA and/or RNA. Such pathogen disinfection is useful in: (a) health care applications such as, for example, dermatological and blood disinfection; (b) mold reduction applications; (c) air and water purification applications; (d) food processing and preservation applications; (e) food and beverage packaging decontamination applications; (f) UV curing of paints and glues applications; (g) HVAC applications in hospitals, businesses and schools to reduce mold, bacteria and pathogens, for example, in AC ducts; and (h) grow light applications for horticulture.

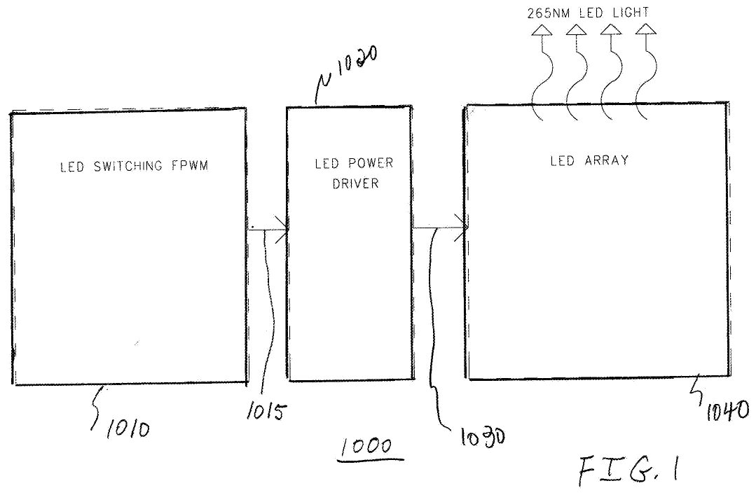

FIG. 1 shows a block diagram of FPWM modulated UVC LED array apparatus 1000 that is fabricated in accordance with one or more embodiments. As shown in FIG. 1, FPWM modulated UVC LED array apparatus 1000 comprises FPWM signal generator 1010 which produces FPWM output signal 1015. As further shown in FIG. 1, FPWM output signal 1015 is applied as input to LED power driver 1020, and in response, LED power driver 1020 outputs FPWM, LED power drive signal 1030. As still further shown in FIG. 1, FPWM LED power drive signal 1030 is applied as input to LED array 1040, and in response, LED array 1040 outputs FPWM modulated UVC radiation.

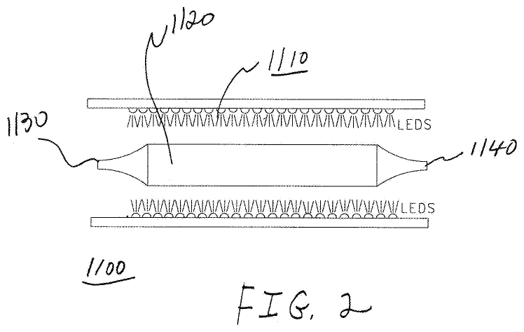

FIG. 2 shows a cross section 1100 of a portion of FPWM modulated UVC LED array apparatus 1000 that is fabricated in accordance with one or more embodiments in use in an application to disinfect a liquid such as, for example and without limitation, blood, wherein an LED array surrounds a cuvette through which the liquid may pass. As shown in FIG. 2, cross section 1100 of FPWM modulated UVC LED array apparatus 1000 comprises LED array 1110 which transmits radiation into cuvette 1120. In accordance with one or more embodiments, cuvette 1120 is substantially transparent to UVC radiation and ends 1130 and 140 have apertures so that liquid may enter at aperture 1130 and exit through aperture 1140. Any one of a number of configurations of cuvette 1120 may be suitable for use in a particular application such as, for example and without limitation, tubular. In accordance with one or more embodiments, cuvette 1120 is comprised of fused silica quartz or any one of a number of materials that are well known to those of ordinary skill in the art.

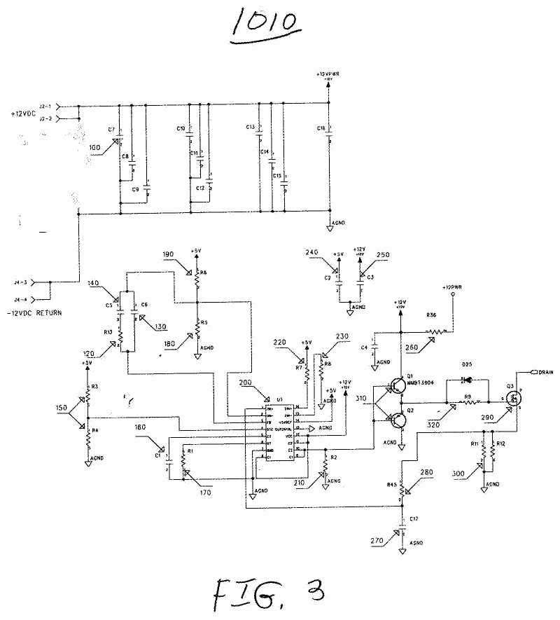

FIG. 3 shows a schematic diagram of FPWM signal generator 1010 that is fabricated in accordance with one or more embodiments. In accordance with one or more embodiments, output from FPWM signal generator 1010 has a frequency of 1±5% kHz and a pulse width of 100±2% pec for a duty cycle which is about 10%. Such parameters advantageously result in LED lifetimes in a range between about 50K and about 100K hours. In addition, such parameters enable the LED array to operate by providing high intensity pulses while cooling is provided by convection.

As shown in FIG. 3, arrow 100 refers to a charge storage system comprised of capacitors C7-C16. In accordance with one or more embodiments, as described below, discharge of the charge storage system supplies current pulses that drive the LED array. The arrangement shown in FIG. 3 advantageously enables the use of ceramic capacitors to fabricate the storage system. In accordance with one or more embodiments, each of capacitors C7-C16 is a 100 μF @ 16V ceramic capacitor to provide a high current pulse. In accordance with one or more embodiments, AGND is an “analog ground reference” which is generated by FPWM control circuit U1 200. In addition, as shown in FIG. 3, a 12 VDC power supply (not shown) applies +12 VDC to one end of the storage system (via jacks J2-1 and J2-2) and −12 VDC to the other end of the storage system (via jacks J4-1 and J4-2).

As further shown in FIG. 3, arrow 150 refers to a voltage divider used to set up the pulse width of the FPWM signal, which voltage divider is comprised of resistors R3 and R4. As shown in FIG. 3, one end of resistor R3 is connected to +5 VDC. The other end of resistor R3 is connected to: (a) one end of resistor R4 and (b) pin 4 of FPWM control circuit U1 200. The functions provided by FPWM control circuit U1 200 will be described below. In accordance with one or more embodiments, FPWM control circuit U1 200 receives +12 VDC as input, and in accordance with one of its functions, outputs a +5 VDC signal which is applied to resistor R3. In accordance with one or more embodiments, FPWM control circuit U1 200 uses input at pin 4 from voltage divider 150 to provide the pulse width of the FPWM signal applied to the LED array. A method for determining values of resistors R3 and R4 suitable to provide a 100 μsec pulse width is well known. In accordance with one or more embodiments, the value of resistor R3 is 2.80KΩ· 1/10 W and the value of resistor R4 is 2.26KΩ·⅕ W.

As further shown in FIG. 3, arrow 160 refers to timing capacitor C1 and arrow 170 refers to timing resistor R1. As shown in FIG. 3, one end of timing capacitor C1 is connected to one end of timing resistor R1 and to AGND at pin 7 of FPWM control circuit U1 200. The other end of timing capacitor C1 is connected to pin 5 of FPWM control circuit U1 200 and the other end of resistor R1 is connected to pin 6 of FPWM control circuit U1 200. FPWM control circuit U1 200 uses the input from timing resistor R1 and the input from timing capacitor C1 to set the FPWM frequency. A method for determining values of timing capacitor C1 and timing resistor R1 to set the FPWM frequency is well known. In accordance with one or more embodiments, the value of capacitor C1 is 0.1 μF @ 50V/1% and the value of resistor R1 is P10.0K_0.1% to provide a FPWM frequency of 1 kHz.

As shown in FIG. 3, arrow 120 refers to feedback resistor R13, arrow 130 refers to feedback capacitor C6, arrow 140 refers to feedback capacitor C5, and arrows 180 and 190 refer to a voltage divider comprised of resistors R5 and R6. As shown in FIG. 3, one end of resistor R13 is connected to pin 3 of FPWM control circuit U1 200 and to one end of feedback capacitor C6. The other end of resister R13 is connected to one end of capacitor C5. As further shown in FIG. 3, the other end of capacitor C5 is connected to: (a) the other end of capacitor C6; (b) one end of resistor R5 and one end of resistor R6, and (c) pin 2 of FPWM control circuit U1 200. Further, the other end of resistor R6 is connected to +5 VDC and the other end of resistor R5 is connected to AGND. In accordance with one or more embodiments, the arrangement of capacitor C5, capacitor C6, and resister R13 provides feedback to stabilize the pulse width of the FPWN signal and the voltage divider comprised of resistors R5 and R6 provides peak current feedback. A method for determining suitable values of capacitor C5, capacitor C6, resistor R13, resistor R5 and resistor R6 is well known. In accordance with one or more embodiments, the value of resistor R13 is 4.7KΩ· 1/10 W. In accordance with one or more embodiments, the value of capacitor C6 is 1000 pF @ 50V, the value of capacitor C5 is 0.01 pF @ 50V, and the value of resistors R5 and R6 is each 2.49KΩ· 1/10 W.

As shown in FIG. 3, arrow 200 refers to fixed frequency pulse width modulation control circuit U1 200 (FPWM control circuit U1 200). In accordance with one or more embodiments, FPWM control circuit U1 is a TL494,10 PWM control circuit that is widely available, and suitable sources may be located readily by searching the internet. The TL494 device incorporates all the functions required in the construction of a pulse-width-modulation (PWM) control circuit on a single chip.

The following sets forth the functionality of the TLP494 pins. Pin 1 is named 1IN+ and is a noninverting input to error amplifier 1. Pin 2 is named 1IN− and is an inverting input to error amplifier 1. Pin 3 is named FEEDBACK and is an input pin for feedback. Pin 4 is named DTC and is a dead-time control comparator input. Pin 5 is named CT and is a capacitor terminal used to set oscillator frequency. Pin 6 is named RT and is a resistor terminal used to set oscillator frequency. Pin 7 is named GND and is a ground. Pin 8 is named C1 and is a collector terminal of BJT output 1. Pin 9 is named E1 and is an emitter terminal of BJT output 1. Pin 10 is named E2 and is an emitter terminal of BJT output 2. Pin 11 is named C2 and is a collector terminal of BJT output 2. Pin 12 is named V_{CC} and is a positive supply. Pin 13 is named OUTPUT CTRL and selects single-ended/parallel output or push-pull operation. For parallel operation, the output-control input must be grounded. This disables the pulse-steering flip-flop and inhibits its outputs. In this mode, the pulses seen at the output of a dead-time control/PWM comparator are transmitted by both output transistors in parallel. Pin 14 is named REF and is a 5-V reference regulator output. Pin 15 is named 2IN− and is inverting input to error amplifier 2. Pin 16 is named 2IN+ and is noninverting input to error amplifier 2.

As further shown in FIG. 3, arrow 210 refers to common emitter resistor R2 used for PWM square wave output produced at pins 9 and 10 of FPWM control circuit U1 200. As shown in FIG. 3, one end of resistor R2 is connected to AGND and the other end of resistor R2 is connected to pins 9 and 10 of FPWM control circuit U1 200 and to the base of npn transistor Q1 and the base of pnp transistor Q2 of complementary symmetry transistor gate driver 310. The emitters of transistors Q1 and Q2 are connected to each other. A method for determining a suitable value for resistor R2 is well known. In accordance with one or more embodiments, the value of resistor R2 is 499Ω·¼ W, 1%, transistor Q1 is an MMBT3904 transistor and transistor Q2 is an MMBT3906 transistor, which transistors are widely available, and suitable sources may be located readily by searching the internet.

As further shown in FIG. 3, arrow 220 refers to OP AMP #2 (of FPWM control circuit U1 200) pull-up resistor R7, and arrow 230 refers to OP AMP #2 pull-down resistor R8. As shown in FIG. 3, resistor R7 is connected at one end to +5 VDC and is connected at its other end to pin 16 of FPWM control circuit U1 200 and resistor R8 is connected at one end to AGND and is connected at its other end to pin 15 of FPWM control circuit U1 200. A method for determining suitable values of resistors R7 and R8 is well known. In accordance with one or more embodiments, the value of resistors R7 and R8 are each 2.49KΩ· 1/10 W.

As further shown in FIG. 3, arrow 240 refers to 5 VDC filter capacitor C2, and arrow 250 refers to 12 VDC filter capacitor C3. The manufacturer of FPWM control circuit U1 200 recommends the usage of these filter capacitors for stability. A method for determining suitable values of capacitors C2 and C3 is well known. In accordance with one or more embodiments, the value of capacitors C2 and C3 are each 1 μF @ 25V.

As further shown in FIG. 3, arrow 260 refers to a ripple reduction circuit comprised of resistor R36 and capacitor C4. As shown in FIG. 3, one end of resistor R36 is connected to a 12V bus and the other end of resistor R36 is connected to: (a)+12 VDC; (b) one end of capacitor C4; and (c) the collector of npn transistor Q1 of push-pull circuit 310. A method for determining suitable values of resistor R36 and capacitor C4 is well known. In accordance with one or more embodiments, the value of resistor R36 is 5.1 Ω·⅛ W 5% and the value of C4 is 22 μF @ 25V.

As further shown in FIG. 3, arrow 270 refers to current ripple reduction circuit comprised of resistor R45 and capacitor C17, which current ripple reduction circuit is used for accurate tracking. As shown in FIG. 3, one end of capacitor C17 is connected to AGND and the other end of capacitor C17 is connected to one end of resistor R45 and to pin 1 of FPWM control circuit U1 200. The other end of resistor R45 is connected to one end of each of current sense resistors R11 and R12, and to the source of main power switching MOSFET Q3. The other end of each of current sense resistors R11 and R12 is connected to AGND. A method for determining suitable values of resistors R45, R11 and R12, and capacitor C17 is well known. In accordance with one or more embodiments, the value of capacitor C17 is 0.1 μF @ 16V, the value of resistor R45 is 2.49KΩ· 1/10 W and the value of each of resistors R11 and R12 is 0.212.7 W. In accordance with one or more embodiments, Q3 is an NTD5C648NLT4G MOSFET which is widely available, and suitable sources may be located readily by searching the internet.

As further shown in FIG. 3, arrow 320 refers to current limiting gate drive resistor R9. As shown in FIG. 3, the emitters of transistors Q1 and Q2 are connected to one end of resistor R9 and to the cathode of diode D25. The other end of resistor R9 is connected to the anode of diode D25 and to the gate of MOSFET Q3. Resistor R9 and diode D25 function as a current limiting device that protects the gate of power MOSFET Q3 and to balance the load. A method for determining a suitable value of resistor R9 and a suitable diode for diode D25 is well known. In one embodiment, the value of resistor R9 is 5.1Ω·⅛ W and diode D25 is a 1N4148 W-TP diode which is widely available, and suitable sources may be located readily by searching the internet.

An LED array drive signal is supplied as output from the drain of MOSFET Q3.

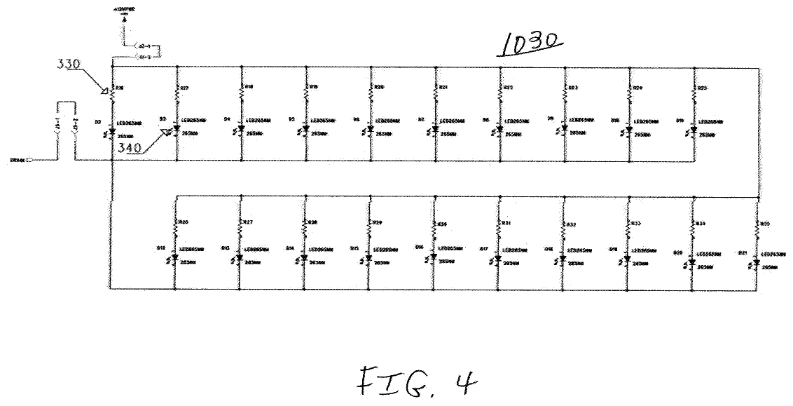

FIG. 4 shows a schematic diagram of LED array 1030 that is fabricated in accordance with one or more embodiments. As shown in FIG. 4, UVC LEDs D2-D21 are arranged in parallel and each of them is in series with one of balancing resistors R16-R33, respectively. Advantageously, resistors R16-R33 distribute current over the array of LEDs and allow adjustment of LED intensity. The current for each LED is tuned using the resistors. In accordance with one or more embodiments, each of the LEDs is a nominal 265 nm LED (i.e., output is 265 nm±5 nm) that outputs radiation in a narrow band of wavelengths centered substantially around 265 nm and having a divergence angle of approximately 30°, and the value of each of resistors R16-R33 is 1.0Ω·¼ W. In accordance with one embodiment, the LEDs are 265 nm deep UV LEDs which are widely available, and suitable sources may be located readily by searching the internet. In accordance with one or more embodiments, the LEDs are pn #HYF38P40F250AG-S, made by Shenzhen-YingFeng Opto-Electronics of China. In accordance with one or more embodiments, the FPWM modulated LED array apparatus develops a high current and low voltage pulse which produces a high intensity UVC beam of radiation.

The FPWM modulated LED array apparatus described above may be used in any number of applications, as mentioned above. For example, a disinfection apparatus would include a chamber in which a sample to be disinfected is placed. Such an apparatus would include power-on-off switches for the FPWM modulated LED array apparatus, which power-on-off switches operate in conjunction with timers that enable one to set an exposure time. For a liquid sample application, the chamber would be, for example and without limitation, the cuvette shown in FIG. 2. Such apparatus would include, for example, pumps to inject the liquid into the cuvette and pumps to retrieve the liquid after disinfection.

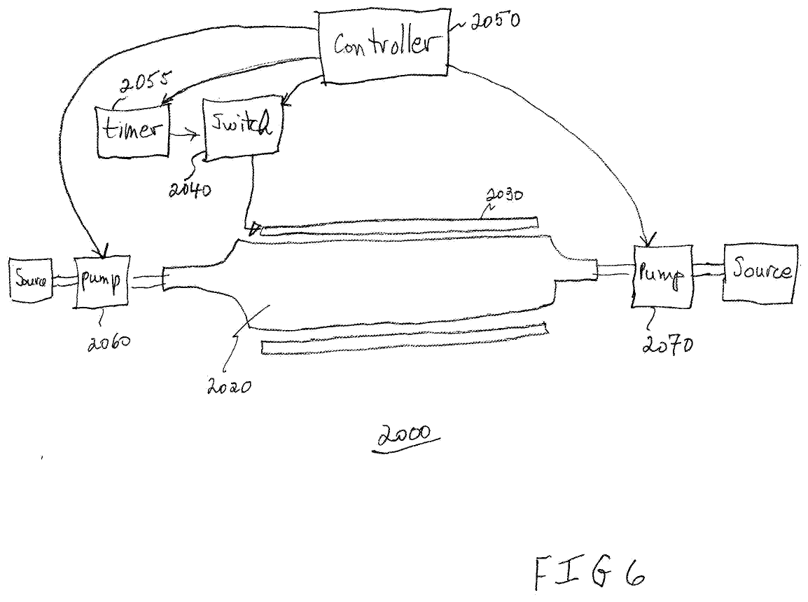

FIG. 6 shows an embodiment of an FPWM modulated LED array apparatus that is used in blood disinfection apparatus 2000. As shown in FIG. 6, blood disinfection apparatus 2000 includes chamber 2020 (for example, cuvette 1120 shown in FIG. 2). As further shown in FIG. 6, FPWM modulated LED array apparatus 2030 (for example, FPWM modulated LED array apparatus 1110 shown in FIG. 2) is disposed about the surface of cuvette 2020 so that, upon energization, radiation output from FPWM modulated LED array apparatus 2030 is transmitted into cuvette 1120. As further shown in FIG. 6, power-on-off switch 2040 is connected to FPWM modulated LED array apparatus 2030 in conjunction with timer 2055 to enable one to set an exposure time. In accordance with one or more embodiments, power-on-off switch 2040 is an on/off key switch available from OMRON as pn #A22NK-2BM-01BA-G100. As further shown in FIG. 6, pumps 2060 are used to inject blood into cuvette 2020 and pumps 2070 are used to retrieve the blood after a period of disinfection. Lastly, blood disinfection apparatus 2000 includes controller 2055 (for example, a microprocessor) that directs the action of power-on-off switch 2040, timer 2055, and pumps 2060 and 2070. In accordance with one or more embodiments, a suitable power-on-off switch, a suitable timer (such as, for example and without limitation, a programmable timer cycle switch from ICstation pn #12V10A Timer Module), and suitable pumps may be readily determined by one of ordinary skill in the art. In addition, in accordance with one or more embodiments, FPWM modulated LED array apparatus 2030 is fabricated in accordance with the description set forth herein.

Advantages of various embodiments are: (a) due to the high current pulse of the inventive FPWM modulated LED array apparatus, a high level of intensity (i.e., the power of radiant energy per unit area measured as mW/cm² (milli-watts/cm²) or measured as mJ/sec-cm² (milli-Joules/sec-cm²)) can be achieved which is more than ten times the intensity of a conventional LED array, and as a result, for example, UV curing and pathogen eradication exposure times are substantially reduced. In accordance with one or more embodiments, the flux can be as high as 20 mW/cm². Thus, the FPWM modulated LED array apparatus: (i) increases intensity to a such a level that it can compete with low pressure mercury lamps, (ii) increases efficiency over a mercury lamp by 50%, (iii) increases the life of LEDs to over 50,000 hours, (iv) increases the efficacy of LED output, i.e., the power to provide a desired effect, by over 50%, (v) increases the mean time between failure (“MTBF”) of the LED array to over 50,000 hours, (vi) increases production throughput due to increased UVC intensity; (vii) can include optics to reduce angular divergence of LED output and, thereby increase the intensity.

In accordance with one or more embodiments, the LED beams of the array are interleaved to provide uniform output. In addition, the divergence angle of the LED beams may be altered by the use of lenses which are affixed to the LEDs. In addition, suitable divergence angles may be used to inhibit the occurrence of standing waves due to reflection of the LED beams, which standing waves would produce “dead spots” that might tend to reduce the intensity of the beams impinging upon the material to be irradiated.

Embodiments described above are exemplary. As such, many changes and modifications may be made to the description set forth above by those of ordinary skill in the art while remaining within the scope of the invention. In addition, materials, methods, and mechanisms suitable for fabricating embodiments have been described above by providing specific, non-limiting examples and/or by relying on the knowledge of one of ordinary skill in the art. Materials, methods, and mechanisms suitable for fabricating various embodiments or portions of various embodiments described above have not been repeated, for sake of brevity, wherever it should be well understood by those of ordinary skill in the art that the various embodiments or portions of the various embodiments could be fabricated utilizing the same or similar previously described materials, methods or mechanisms. As such, the scope of the invention should be determined with reference to the appended claims along with their full scope of equivalents.

What is claimed is:

1. A modulated LED array apparatus comprises:
a modulation signal generator that produces a pulse width modulation (PWM) signal;
the PWM signal is applied as input to an LED power drive circuit that produces an LED power drive signal;
the LED power drive signal is applied as input to an array of LEDs and, in response, the LED array outputs PWM radiation;
wherein the LEDs in the array output radiation in a predetermined band of wavelength.

2. The modulated LED array apparatus of claim 1 wherein:
the LED power drive circuit includes a charge storage assembly comprised of capacitors.

3. The modulated LED array apparatus of claim 2 wherein the LEDs of the array are arranged in parallel and are arranged so that radiation output from neighbors of LEDs is interleaved to provide a substantially uniform beam.

4. The modulated LED array apparatus of claim 1 wherein the predetermined band of wavelength includes UVC radiation having a wavelength at about 265 nm.

5. The modulated LED array apparatus of claim 4 wherein:
the modulation signal generator is a fixed frequency, pulse width modulation (FPWM) signal generator.

6. The modulated LED array apparatus of claim 5 wherein FPWM output from the FPWM signal generator has a frequency at about 1 KHz and a pulse width of about 100 μsec for a duty cycle of about 10%.

7. The modulated LED array apparatus of claim 6 wherein the charge storage assembly is comprised of a parallel arrangement of ceramic capacitors.

8. The modulated LED array apparatus of claim 7 wherein the capacitors in the charge storage assembly are 100 μF @ 16V ceramic capacitors.

9. The modulated LED array apparatus of claim 8 wherein each of the LEDs in the LED array is in series with a balancing resistor.

10. The modulated LED array apparatus of claim 9 wherein the output from the LED array is arranged to inhibit the occurrence of standing waves due to reflection of the LED output.
